(19) United States
(12) Patent Application Publication
(10) Pub. No.: US 2004/0092558 A1
(43) Pub. Date: May 13, 2004

(54) Histone deacetylase inhibitors for the treatment of ocular neovascular or edematous disorders and diseases

(75) Inventors: Peter G. Klimko, Fort Worth, TX (US); David P. Bingaman, Fort Worth, TX (US)

Correspondence Address: Alcon Research, Ltd., Teresa J. Schultz, Q-148, 6201 South Freeway, Fort Worth, TX, 76134-2099, US

(73) Assignee: Alcon, Inc., Hunenberg (CH)

(21) Appl. No.: 10/697,135

(22) Filed: Oct. 30, 2003

Related U.S. Application Data
(60) Provisional application No. 60/425,574, filed on Nov. 12, 2002.

(51) Int. Cl. A61K 31/44; A61K 31/40; A61K 31/19

(52) U.S. Cl. 514/357; 514/408; 514/575

(57) ABSTRACT

Ophthalmic compositions containing HDAC inhibitors and their use for treating ocular neovascular or edematous diseases and disorders are disclosed.

### BACKGROUND OF THE INVENTION

This application claims priority from U.S. S. No. 60/425,574, filed Nov. 12, 2002.

The present invention is directed to histone deacetylase (HDAC) inhibitors in ophthalmic compositions and their methods of use. The compounds are particularly useful in treating persons suffering from an ocular neovascular or edematous disease or disorder.

### BACKGROUND OF THE INVENTION

There are many agents known to inhibit the formation of new blood vessels (angiogenesis). For example, steroids functioning to inhibit angiogenesis in the presence of heparin or specific heparin fragments are disclosed in Crum, et al., A New Class of Steroids Inhibits Angiogenesis in the Presence of Heparin or a Heparin Fragment, Science, Vol. 230:1375-1378, Dec. 20, 1985. The authors refer to such steroids as “angiostatic” steroids. Included within this class of steroids found to be angiostatic are the dihydro and tetrahydro metabolites of cortisol and cortexolone. In a follow-up study directed to testing a hypothesis as to the mechanism by which the steroids inhibit angiogenesis, it was shown that heparin/angiostatic steroid compositions cause dissolution of the basement membrane scaffolding to which anchorage dependent endothelia are attached resulting in capillary involution; see, Ingber, et al., A Possible Mechanism for Inhibition of Angiogenesis by Angiostatic Steroids: Induction of Capillary Basement Membrane Dissolution, Endocrinology Vol. 119:1768-1775, 1986.

A group of tetrahydro steroids useful in inhibiting angiogenesis is disclosed in U.S. Pat. No. 4,975,537, Aristoff, et al. The compounds are disclosed for use in treating head trauma, spinal trauma, septic or traumatic shock, stroke, and hemorrhage shock. In addition, the patent discusses the utility of these compounds in embryo implantation and in the treatment of cancer, arthritis, and arteriosclerosis. Some of the steroids disclosed in Aristoff et al. are disclosed in U.S. Pat. No. 4,771,042 in combination with heparin or a heparin fragment for inhibiting angiogenesis in a warm blooded animal.

Compositions of hydrocortisone, “tetrahydrocortisol-S,” and U-72,745G, each in combination with a beta cyclodextrin, have been shown to inhibit corneal neovascularization: Li, et al., Angiostatic Steroids Potentiated by Sulphated Cyclodextrin Inhibit Corneal Neovascularization, Investigative Ophthalmology and Visual Science, Vol. 32(11):2898-2905, October, 1991. The steroids alone reduce neovascularization somewhat but are not effective alone in effecting regression of neovascularization.

Tetrahydrocortisol (THF) has been disclosed as an angiostatic steroid in Folkman, et al., Angiostatic Steroids, Ann. Surg., Vol. 206(3), 1987, wherein it is suggested angiostatic steroids may have potential use for diseases dominated by abnormal neovascularization, including diabetic retinopathy, neovascular glaucoma, and retrolental fibroplasia.

It has been previously shown that certain nonsteroidal anti-inflammatory drugs (NSAIDs) can inhibit angiogenesis and vascular edema in pathologic conditions. The ability of most NSAIDs to influence vascular permeability, leading to edema, and angiogenesis appears to be associated with their ability to block the cyclo-oxygenase enzymes (COX-1 and -2). Blockade of COX-1 and -2 is associated with a decrease in inflammatory mediators, such as PGE₂. Moreover, it appears that PGE₂ inhibition results in decreased expression and production of various cytokines including vascular endothelial growth factor (VEGF). VEGF is known to produce vascular leakage and angiogenesis in the eye of pre-clinical models. Also, increased levels of VEGF have been found in neovascular tissues and extracellular fluid from the eyes of patients with diabetic retinopathy and age-related macular degeneration. Thus, NSAIDs may inhibit vascular leakage and angiogenesis by modulating PGE₂ levels and its effects on VEGF expression and activity. This theory is supported by work involving animal tumor models which demonstrate that systemic administration of COX-2 inhibitors decreases PGE₂ and VEGF tissue levels and thereby prevents tumor-induced angiogenesis. In these models, VEGF activity and angiogenesis are restored by adding exogenous PGE₂ during continued COX-2 blockade. However, NSAIDs appear to have variable activity in animal models of ocular neovascularization (NV), in that selective COX inhibitors do not appear to inhibit choroidal neovascularization. In fact, these studies have called into question the role of COX-1 and/or COX-2 in the development of CNV.

As described in commonly owned U.S. application Ser. No. 09/929,381, it was found that certain 3-benzoylphenlacetic acids and derivatives, which are NSAIDs, are useful for treating angiogenesis-related disorders.

Histones are nuclear proteins that form octameric particles around which chromosomal DNA is wound in a repeating fashion. This DNA storage mode helps to fit extremely long DNA molecules in the nucleus, helps to stabilize DNA against damage, and serves to regulate the accessibility of DNA to transcription factors. Histones have long, positively charged lysine tails that are electrostatically attracted to the negatively charged phosphate backbone of DNA, thus serving to form the DNA-histone complex. In this state transcription factors do not have access to DNA, and therefore gene expression is repressed. Acetylation of the lysine nitrogens causes local unwinding of the DNA-histone complex and allows transcription factor access, thus facilitating gene expression. The histone deacetylase (HDAC) enzyme family catalyze the conversion of Nacetylated lysines back to the unacetylated state, causing re-formation of the histone-DNA complex and thus repress gene transcription.

One theory as to the oncogenic transformation of a cell posits the importance of the imbalance between pro-oncogenic and anti-oncogenic signals. More specifically, loss of function mutations in genes coding for tumor suppressor proteins, such as p53 and p21, have been correlated with cancer progression. Agents that promote the expression of tumor suppressor proteins and/or induce poorly differentiated cancer cells to undergo differentiation are the subject of many approaches to cancer therapy.

The HDAC enzyme family, by repressing gene transcription, repress the expression of pro-differentiation and tumor-suppressor proteins. Thus inhibition of this enzyme family is being investigated as an anti-cancer therapeutic strategy. In particular, several HDAC inhibitors have shown promise in pre-clinical models of various cancers. For example, the HDAC inhibitor suberoylanilide hydroxamic acid (SAHA) has been reported to be a potent inducer of cancer cell differentiation (Munster et. al., Cancer Research, Vol. 61:8492-8497, 2001), to arrest cancer cell growth in vitro (Butler et. al., Proc. Natl. Acad. Sci. USA, Vol. 99:11700-11705, 2002), to shrink tumors in animal models (Butler et. al., Cancer Res., Vol. 60:5165-5170, 2000) showed almost no dose-limiting toxicity in phase I clinical trials including no suppression of white blood cell production, which is very unusual for an anticancer agent (Kelly et. al., Proc. Amer. Soc. Clin. Oncol., Vol. 20:87a, 2001), and is currently in phase 11 clinical trials. Furthermore, it has been recently shown that HDAC enzyme activity promotes angiogenesis by inhibiting tumor suppressor protein expression (Kim et. al., Nature Medicine, Vol. 7:437-443, 2001) and that HDAC inhibitors, including SAHA, can inhibit VEGF-induced neovascularization (Deroanne et. al., Oncogene, Vol. 21:427-436, 2002).

### SUMMARY OF THE INVENTION

The present invention is directed to the use of HDAC inhibitors to treat persons suffering from an ocular neovascular or edematous disease or disorder.

### DETAILED DESCRIPTION OF THE INVENTION

Posterior segment neovascularization is the vision-threatening pathology responsible for the two most common causes of acquired blindness in developed countries: exudative age-related macular degeneration (AMD) and proliferative diabetic retinopathy (PDR). Currently the only approved treatments for the posterior segment NV that occurs during exudative AMD are laser photocoagulation or photodynamic therapy with Visudyne®; both therapies involve occlusion of affected vasculature which results in localized laser-induced damage to the retina. Surgical interventions with vitrectomy and membrane removal are the only options currently available for patients with proliferative diabetic retinopathy. No strictly pharmacologic treatment has been approved for use against posterior segment NV, although several different compounds are being evaluated clinically, including, for example, anecortave acetate (Alcon, Inc.), EYE 001 (Eyetech), and rhuFabV2 (Genentech) for AMD and LY333531 (Lilly) and Fluocinolone (Bausch & Lomb) for diabetic macular edema.

In addition to changes in the retinal microvasculature induced by hyperglycemia in diabetic patients leading to macular edema, proliferation of neovascular membranes is also associated with vascular leakage and edema of the retina. Where edema involves the macula, visual acuity worsens. In diabetic retinopathy, macular edema is the major cause of vision loss. Like angiogenic disorders, laser photocoagulation is used to stabilize or resolve the edematous condition. While reducing further development of edema, laser photocoagulation is a cytodestructive procedure, that, unfortunately will alter the visual field of the affected eye.

An effective pharmacologic therapy for ocular NV and edema would likely provide substantial efficacy to the patient, in many diseases thereby avoiding invasive surgical or damaging laser procedures. Effective treatment of the NV and edema would improve the patient's quality of life and productivity within society. Also, societal costs associated with providing assistance and health care to the blind could be dramatically reduced.

It is believed that HDAC inhibitors (Compounds) among other utilities, inhibit VEGF induced neovascularization and are therefore useful for treating a human patient suffering from an ocular NV or edematous disease or disorder, such as, diabetic retinopathy, chronic glaucoma, retinal detachment, sickle cell retinopathy, age-related macular degeneration, rubeosis iritis, uveitis, neoplasms, Fuch's heterochromic iridocyclitis, neovascular glaucoma, corneal neovascularization, neovascularization resulting from combined vitrectomy and lensectomy, retinal ischemia, choroidal vascular insufficiency, choroidal thrombosis, carotid artery ischemia, contusive ocular injury, retinopathy of prematurity, retinal vein occlusion, proliferative vitreoretinopathy, corneal angiogenesis, retinal microvasculopathy, and retinal (macular) edema. They are particularly attractive given the low mechanism-related toxicity (for reviews on the classes of compounds which function as HDAC inhibitors and are being investigated for oncology applications, see: Marks et. al., Nature Reviews Cancer, Vol. 1:194-202, 2001; Marks et. al., Curr. Opin. Oncol., Vol. 13:477-483, 2001).

Particularly preferred HDAC inhibitors of the present invention include those of formula I

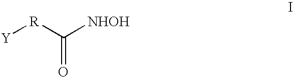

wherein:

Y=R¹NHC(O) or R²C(O)NR³;

R²=an optionally substituted aryl, heteroaryl, cycloalkyl, heterocycloalkyl, aryloxy, arylalkyloxy, or alkyl, where the aryl, etc. cyclic systems can be bicyclic;

R²=an optionally substituted aryl, heteroaryl, cycloalkyl, heterocycloalkyl, aryloxy, arylalkyloxy, or alkyl, where the aryl, etc. cyclic systems can be bicyclic;

R³=H, alkyl, or C(O)R⁴;

R⁴=an optionally substituted aryl, heteroaryl, cycloalkyl, heterocycloalkyl, aryloxy, arylalkyloxy, or alkyl, where the aryl, etc. cyclic systems can be bicyclic;

R=(CH₂)ₙ or CH(A-R⁵)—(CH₂)ₙ₋₁;

n=3-8;

A=NH, O, S, CH₂, NHCO, or NHCO₂; and

R⁵=an optionally substituted aryl, heteroaryl, cycloalkyl, heterocycloalkyl, or alkyl, where the aryl, etc. cyclic systems can be bicyclic.

Included among the specifically preferred compounds of the present invention formula I are the following compounds:

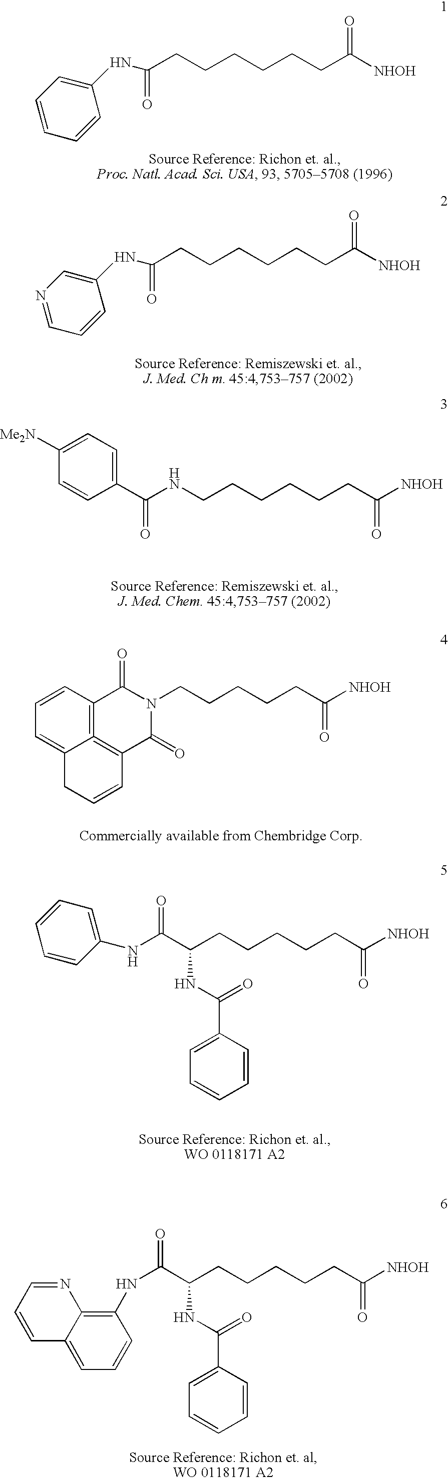

Compounds 1-3, 5, and 6 can be synthesized by methods detailed in the source references. Compound 4 is commercially available from the Chembridge Corporation, 16981 Via Tazon, Suite G, San Diego, Calif., USA, 92127.

Other specifically preferred compounds of the present invention include the following compounds:

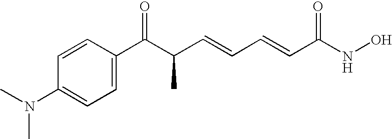

Trichostatin A, Commercially available from Sigma, PO Box 14508, St. Louis, Mo., 63178-9916

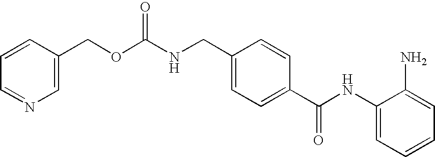

MS-275:. Source Reference: Suzuki et. al., J. Med. Chem., 42:15, 3001-3003 (1999).

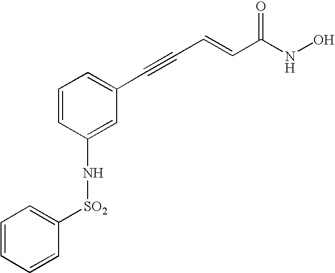

Oxamflatin: Commercially available from Calbiochem-Novabiochem International, 10394 Pacific Center Court, San Diego, Calif. 92121, USA Included within the scope of the present invention are the individual enantiomers of the title compounds, as well as their racemic and non-racemic mixtures. Generally, the individual enantiomers can be procured by a number of methods, including but not limited to: enantioselective synthesis from the appropriate enantiomerically pure or enriched starting material; synthesis from racemic/non-racemic or achiral starting materials using a chiral reagent, catalyst, solvent, etc. (see for example: Asymmetric Synthesis, J. D. Morrison and J. W. Scott, Eds. Academic Press Publishers, (New York) 1985), volumes 1-5; Principles of Asymmetric Synthesis, R. E. Gawley and J. Aube, Eds.; Elsevier Publishers (Amsterdam 1996)); and isolation from racemic and non-racemic mixtures by a number of known methods, e.g. by purification of a sample by chiral HPLC (A Practical Guide to Chiral Separations by HPLC, G. Subramanian, Ed., VCH Publishers, (New York 1994); Chiral Separations by HPLC, A. M. Krstulovic, Ed., Ellis Horwood Ltd. Publishers (1989)), or by enantioselective hydrolysis of a carboxylic acid ester sample by an enzyme (Ohno, M.; Otsuka, M., Organic Reactions, 37:1 (1989)). Those skilled in the art will appreciate that racemic and non-racemic mixtures may be obtained by several means, including without limitation, nonenantioselective synthesis, partial resolution, or even mixing samples having different enantiomeric ratios. Departures may be made from such details within the scope of the accompanying claims without departing from the principles of the invention and without sacrificing its advantages. Also included within the scope of the present invention are the individual isomers substantially free of their respective enantiomers.

The term “alkyl” includes straight or branched chain aliphatic hydrocarbon groups that are saturated and have 1 to 15 carbon atoms. The alkyl groups may be substituted with other groups, such as halogen, hydroxyl or alkoxy. Preferred straight or branched alkyl groups include methyl, ethyl, propyl, isopropyl, butyl and t-butyl.

The term “cycloalkyl” includes straight or branched chain, saturated or unsaturated aliphatic hydrocarbon groups which connect to form one or more rings, which can be fused or isolated. The rings may be substituted with other groups, such as halogen, amino, hydroxyl, alkoxy, or lower alkyl. Preferred cycloalkyl groups include cyclopropyl, cyclobutyl, cyclopentyl and cyclohexyl.

The term “heterocycloalkyl” refers to cycloalkyl groups which contain at least one heteroatom such as O, S, or N in the ring. Heterocycloalkenyl rings may be isolated, with 5 to 8 ring atoms, or fused, with 8 to 10 atoms. The heterocycloalkyl ring(s) hydrogens or heteroatoms with open valency may be substituted with other groups, such as lower alkyl, acyl, amino, hydroxy, or halogen. Preferred heterocycloalkyl groups include piperidine, piperazine, pyrrolidine, tetrahydrofuranyl, tetrahydropyranyl, and tetrahydrothienyl.

The term “lower alkyl” represents alkyl groups containing one to six carbons (C₁-C₆).

The term “halogen” represents fluoro, chloro, bromo, or iodo.

The term “aryl” refers to carbon-based rings which are aromatic. The rings may be isolated, such as phenyl, or fused, such as naphthyl. The ring hydrogens may be substituted with other groups, such as lower alkyl, hydroxy, amino, or halogen.

The term “heteroaryl” refers to aromatic hydrocarbon rings which contain at least one heteroatom such as O, S, or N in the ring. Heteroaryl rings may be isolated, with 5 to 6 ring atoms, or fused, with 8 to 10 atoms. The heteroaryl ring(s) hydrogens or heteroatoms with open valency may be substituted with other groups, such as lower alkyl, amino, hydroxy, or halogen. Examples of heteroaryl groups include imidazole, pyridine, indole, quinoline, furan, thiophene, pyrrole, tetrahydroquinoline, dihydrobenzofuran, and dihydrobenzindole.

The term “aryloxy” refers to an aryl group bonded to an oxygen. The term “arylalkyloxy” refers to an aryl group bonded to an alkyl group, which is bonded to an oxygen atom.

The present invention is also directed to compositions containing Compounds and methods for their use. According to the methods of the present invention, a composition comprising one or more Compounds and a pharmaceutically acceptable carrier for systemic or local administration is administered to a mammal in need thereof. The compositions are formulated in accordance with methods known in the art for the particular route of administration desired.

The Compounds of the present invention can be administered either systemically or locally. Systemic administration includes: oral, transdermal, subdermal, intraperitioneal, subcutaneous, transnasal, sublingual, or rectal. Local administration for ocular administration includes: topical, intravitreal, periocular, transcleral, retrobulbar, sub-tenon, or via an intraocular device. Preferred administration depends on the type of ocular neovascular being treated.

The compositions administered according to the present invention comprise a pharmaceutically effective amount of one or more Compound. As used herein, a “pharmaceutically effective amount” is one which is sufficient to reduce or prevent NV and/or edema. Generally, for compositions intended to be administered systemically for the treatment of ocular NV or edema the total amount of compound will be about 0.01-100 mg/kg.

The following topical ophthalmic and systemic formulations are useful according to the present invention administered 1-4 times per day according to the discretion of a skilled clinician.

### EXAMPLE 1

| Ingredients | Amount (wt %) |
|---|---|
| Compound, especially Compound 1 | 0.01-2% |
| Hydroxypropyl methylcellulose | 0.5% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 2

| Ingredients | Amount (wt %) |
|---|---|
| Compound, especially Compound 2 | 0.01-2% |
| Methyl cellulose | 4.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 3

| Ingredients | Amount (wt %) |
|---|---|
| Compound, especially Compound 3 | 0.01-2% |
| Guar gum | 0.4-6.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 4

| Ingredients | Amount (wt %) |
|---|---|
| Compound, especially Compound 4 | 0.01-2% |
| White petrolatum and mineral oil and | Ointment consistency |
| lanolin | |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |

### EXAMPLE 5

10 mM IV Solution w/v %

| | |
|---|---|
| Compound, especially Compound 5 | 0.384% |
| L-Tartaric acid | 2.31% |
| Sodium hydroxide | pH 3.8 |
| Hydrochloric acid | pH 3.8 |
| Purified water | q.s. 100% |

### EXAMPLE 6

5 mg Capsules

| | mg/capsule |
|---|---|
| Ingredient | (Total Wt. 22a? mg) |
| Compound, especially Compound 6 | 5 |
| Lactose, anhydrous | 55.7 |
| Starch, Sodium carboxy-methyl | 8 |
| Cellulose, microcrystalline | 30 |
| Colloidal silicon dioxide | .5 |
| Magnesium stearate | .8 |

The preferred compositions of the present invention are intended for administration to a human patient suffering from an ocular NV or edematous disease or disorder, such as, diabetic retinopathy, chronic glaucoma, retinal detachment, sickle cell retinopathy, age-related macular degeneration, rubeosis iritis, uveitis, neoplasms, Fuch's heterochromic iridocyclitis, neovascular glaucoma, corneal neovascularization, neovascularization resulting from combined vitrectomy and lensectomy, retinal ischemia, choroidal vascular insufficiency, choroidal thrombosis, carotid artery ischemia, contusive ocular is injury, retinopathy of prematurity, retinal vein occlusion, proliferative vitreoretinopathy, corneal angiogenesis, retinal microvasculopathy, and retinal (macular) edema.

This invention has been described by reference to certain preferred embodiments; however, it should be understood that it may be embodied in other specific forms or variations thereof without departing from its special or essential characteristics. The embodiments described above are therefore considered to be illustrative in all respects and not restrictive, the scope of the invention being indicated by the appended claims rather than by the foregoing description.

We claim:

1. A method for treating persons suffering from an ocular neovascular or edematous disease or disorder which comprises administering a pharmaceutically effective amount of an HDAC inhibitor.

2. The method of claim 1, wherein the HDAC inhibitor is a compound of formula I: 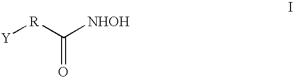
wherein:
Y=R1NHC(O) or R2C(O)NR3;
R¹=an optionally substituted aryl, heteroaryl, cycloalkyl, heterocycloalkyl, aryloxy, arylalkyloxy, or alkyl, where the aryl, etc. cyclic systems can be bicyclic;
R²=an optionally substituted aryl, heteroaryl, cycloalkyl, heterocycloalkyl, aryloxy, arylalkyloxy, or alkyl, where the aryl, etc. cyclic systems can be bicyclic;
R³=H, alkyl, or C(O)R⁴;
R⁴=an optionally substituted aryl, heteroaryl, cycloalkyl, heterocycloalkyl, aryloxy, arylalkyloxy, or alkyl, where the aryl, etc. cyclic systems can be bicyclic;
R═(CH₂)ₙ or CH(A-R⁵)—(CH₂)ₙ₋₁;
n=3-8;
A=NH, O, S, CH₂, NHCO, or NHCO₂; and
R⁵=an optionally substituted aryl, heteroaryl, cycloalkyl, heterocycloalkyl, or alkyl, where the aryl, etc. cyclic systems can be bicyclic.

3. The method of claim 2, wherein the compound(s) of formula I is(are) selected from the group consisting of:

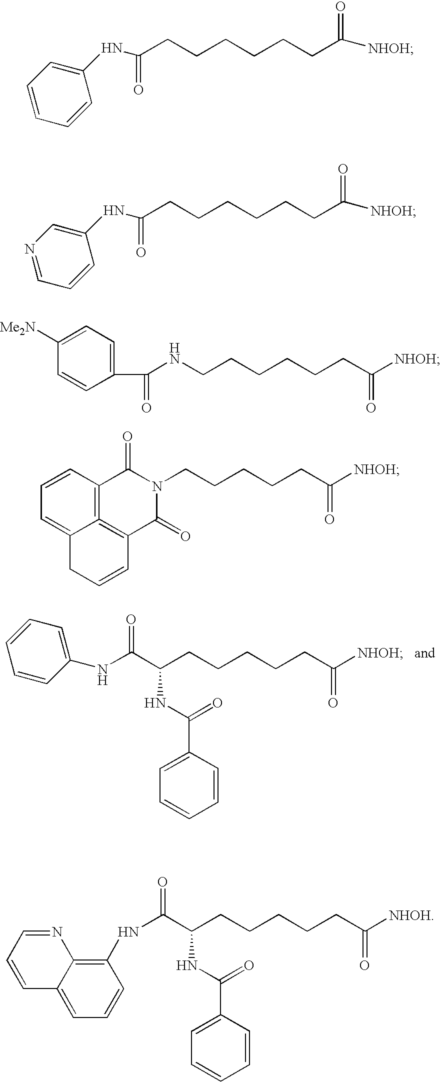

4. The method of claim 1 wherein the ocular neovascular or edematous disease or disorder is selected from the group consisting of diabetic retinopathy, chronic glaucoma, retinal detachment, sickle cell retinopathy, age-related macular degeneration, rubeosis iritis, uveitis, neoplasms, Fuch's heterochromic iridocyclitis, neovascular glaucoma, corneal neovascularization, neovascularization resulting from combined vitrectomy and lensectomy, retinal ischemia, choroidal vascular insufficiency, choroidal thrombosis, carotid artery ischemia, contusive ocular injury, retinopathy of prematurity, retinal vein occlusion, proliferative vitreoretinopathy, corneal angiogenesis, retinal microvasculopathy, and retinal (macular) edema.

5. The method of claim 2 wherein the ocular neovascular or edematous disease or disorder is selected from the group consisting of diabetic retinopathy, chronic glaucoma, retinal detachment, sickle cell retinopathy, age-related macular degeneration, rubeosis iritis, uveitis, neoplasms, Fuch's heterochromic iridocyclitis, neovascular glaucoma, corneal neovascularization, neovascularization resulting from combined vitrectomy and lensectomy, retinal ischemia, choroidal vascular insufficiency, choroidal thrombosis, carotid artery ischemia, contusive ocular injury, retinopathy of prematurity, retinal vein occlusion, proliferative vitreoretinopathy, corneal angiogenesis, retinal microvasculopathy, and retinal (macular) edema.

6. The method of claim 4, wherein the HDAC inhibitor is selected from the group consisting of: [chemical structure]
